(19) United States
(12) Patent Application Publication
(10) Pub. No.: US 2026/0192110 A1
(43) Pub. Date: Jul. 9, 2026

(54) METHOD FOR ESTABLISHING CIRCUIT PROFILE OF ELECTRICAL FIELD SIMULATION FOR TEMPORAL INTERFERENCE BRAIN STIMULATION AND APPLICATION THEREOF

(71) Applicant: NATIONAL YANG MING CHIAO TUNG UNIVERSITY, Hsinchu City (TW)

(72) Inventors: Yu-Ching Chen, Taichung City (TW); Yu-Te Liao, Hsinchu (TW); ALBERT CHIHCHIEH YANG, Hsinchu City (TW)

(21) Appl. No.: 19/091,090

(22) Filed: Mar. 26, 2025

(30) Foreign Application Priority Data
Jan. 7, 2025 (TW) 114100607

(51) Int. Cl. A61N 1/36; G16H 20/40; G16H 30/20

(52) U.S. Cl. CPC A61N 1/36025; G16H 20/40; G16H 30/20

(57) ABSTRACT

A method for establishing circuit model of electrical field simulation for deep brain stimulation in temporal interference includes providing a 3D MRI image, position information of plural electrode members and geometric radius thereof; generating a 3D point array with reference to the 3D MRI image, and the 3D point array constructed by plural cubes aligning with each another; mapping the 3D MRI image, the plural electrode members to set at least source and sink points; establishing a model of brain conductivity distribution with reference to the 3D MRI image is built and a current analysis is done with a circuit simulation software to acquire an equivalent brain circuit profile.

### CROSS REFERENCE

This application claims priority to Taiwan patent application No. 114100607 filed on Jan. 7, 2025, titled as “Method for Establishing Circuit Profile of Electrical Field Simulation for Temporal Interference Brain Stimulation and Application thereof”, all of which are incorporated by reference herein in its entirety.

### FIELD OF THE INVENTION

This present invention relates to the field of non-invasive deep brain stimulation, particularly relates to a system and method for deep brain stimulation with cranial-electro stimulation.

### BACKGROUND OF THE INVENTION

There are repetitive transcranial magnetic stimulation, cranial-electro stimulation, and focused ultrasound fields for current development of brain stimulation techniques. Repetitive transcranial magnetic stimulation changes the electrical activity in the cerebral cortex zone by changing the magnetic field to further affect the functional brain circuit, which has been applied to the treatment of depression nowadays. However, only the superficial cerebral cortex zone is affected by repetitive transcranial magnetic stimulation. Although magnetic stimulation probes for stimulation of deeper brain zones have been developed in medical material technology and used in the treatment of obsessive-compulsive disorder, the development cost of repetitive transcranial magnetic stimulation is relatively higher. Focused ultrasound technology may change the permeability of the blood-brain barrier in specific brain zones by high-energy stimulation to improve medical concentration penetration through the specific brain zone for a good treatment effect. Focused ultrasound technology may also change hemodynamics and brain activities of the specific brain zones by low energy stimulation. However, the cost of developing focused ultrasound technology is also high.

Cranial-electro stimulation is relatively simple compared to repetitive transcranial magnetic stimulation and focused ultrasound technology. Traditional cranial-electro stimulation is not precisive since it only performs current stimulation on non-specific brain or cerebral cortex zones, and it fails in effect on deep brain zones. In recent years, innovative technology of cranial-electro stimulation such as temporal interference brain stimulation has been potential and popular and it has become new methods for non-invasive deep brain stimulation technology. It attracts research groups' attention to explore at a relatively lower cost.

### SUMMARY OF THE INVENTION

Accordingly, a method for establishing circuit profile of electrical field simulation for temporal interference brain stimulation and a formation method for a simulation result of electrical field for temporal interference brain stimulation are provided herein, which may generate an equivalent brain circuit profile with a circuit simulation software and acquire a simulation result by analyzing the equivalent brain circuit profile.

Accordingly, a method for establishing a circuit profile of electrical field simulation for temporal interference brain stimulation includes: providing a three-dimensional MRI image, multiple position information of electrode members and a geometric radius of each of the electrode members; generating a three-dimensional point array with reference to the three-dimensional MRI image, wherein the three-dimensional point array comprises plural nodes on plural cube units, and the plural cube units are spliced together to correspond to the three-dimensional MRI image; set at least a source point and at least a sink point by mapping the three-dimensional point array, the plural position information of electrode members and the plural geometric radiuses to assign some of the plural nodes as at least the source point and at least the sink point; establishing a distribution profile of brain conductivity, wherein the distribution profile of brain conductivity comprises plural resistance data corresponding to the three-dimensional MRI image, and each of the resistance data comprises a resistance value between any two adjacent nodes in the three-dimensional point array; and acquiring an equivalent brain circuit profile by analyzing currents for the distribution profile of brain conductivities, the source point and the sink point with a circuit simulation software.

Accordingly, a formation method for simulation results of an electrical field for temporal interference brain stimulation includes doing an electrical field analysis of the equivalent brain circuit profile to acquire a simulation result of the electrical field.

In one embodiment, the cube units have identical or different sizes.

In one embodiment, the circuit simulation software includes simulation program with integrated circuit emphasis (SPICE) software that executes current analysis with frequencies from 0 Hz to 100 kHz on the distribution profile of brain conductivities.

In one embodiment, the step of doing the electrical field analysis includes calculating a voltage difference between any two adjacent nodes in any one of the cube units and dividing the voltage difference by the distance of the two adjacent nodes to acquire an electrical field value.

In one embodiment, electrode members include two sets of electrode pairs.

Accordingly, a computer-readable storage medium containing instructions executable by a computer for causing the computer to perform operations includes the steps in the method for establishing a circuit profile of electrical field simulation for temporal interference brain stimulation aforementioned.

### BRIEF DESCRIPTION OF THE DRAWINGS

FIG. 1 is a schematic flowchart diagram illustrating an example of a method for establishing a circuit profile of electrical field simulation for temporal interference brain stimulation in accordance with the present invention.

FIG. 2 is a schematic diagram illustrating a three-dimensional point array in accordance with the present invention.

FIG. 3 is a schematic diagram illustrating some content of the database of tissue properties adopted in the present invention.

FIG. 4 is a schematic diagram illustrating the distribution profile of brain conductivities in accordance with the present invention.

FIG. 5 is a schematic flowchart diagram illustrating a method for forming an electrical field simulation of temporal interference brain stimulation in accordance with the present invention.

FIG. 6 is a schematic diagram illustrating an alternating current analysis in the method for forming an electrical field simulation of temporal interference brain stimulation in accordance with the present invention.

### DETAILED DESCRIPTION OF THE PREFERRED EMBODIMENT

A so-called circuit simulation software herein from circuit design fields provides designers with the ability to ensure that the functions they design meet expectations via the circuit simulation software during a design verification. During pre-test design at an earlier stage of manufacturing, there are two simulation profiles of behavior profile: SPICE and IBIS. Simulation Program with Integrated Circuit Emphasis (SPICE) adopts script netlist to describe circuit elements (transistor, resistor and electrical capacitors) and connection therebetween, and find solutions by analyzing nodes and converting nodes to mathematical equations for operation. Behaviors of elements may be predicted with mathematical operations provided by SPICE. Generally, a SPICE profile may duplicate performances of individual elements, which includes passive components such as resistors, electrical capacitors, bipolar diodes, and transistors. These components may appropriately act on the behavior of target elements by suitable designs. There are present SPICE simulators such as LTspice®, NI Multisim™, OrCAD® PSpice Designer and HSPICE, etc.

Next, the technology of temporal interference (TI) stimulation involves imposing electrical stimulation on the brain with two sets of high-frequency alternating currents, which are of different frequencies and different in low frequency fields. The two sets of electrical stimulation are imposed on the brain to respectively generate stimulated electrical fields of high frequencies, and the intercrossing zone between the two stimulated electrical fields of high frequencies forms an envelope modulation field with differences in frequency. The envelope modulation field with differences in frequency is generally called the electrical field of temporal interference.

In the present invention, the position information of electrode members includes deposited position information for each electrode member in the plural sets of electrode pairs that impose alternating currents on the brain. Exemplary position information of electrode member may be described like to: location at left or right side of brain together with distance from superior or inferior external opening of external acoustic meatus, or location at anterior or posterior side of mid-coronal plane or left or right side of mid-sagittal plane together with distance from superior or inferior external opening of external acoustic meatus. Next, a geometric radius of the electrode member represents a size projected by the electrode member on the brain surface. Next, a three-dimensional MRI image is acquired from a physiological signal extracted by magnetic resonance imaging (MRI) to constitute an image with three dimensions. When the three-dimensional MRI image is displayed on a two-dimensional display interface with 2D image tools, it may be sliced or segmented in an appropriate way from a plane such as coronal plane, sagittal plane, or transverse plane for viewing with the 2D image tools. When the three-dimensional MRI image is displayed on a two-dimensional display interface with 3D image tools, a user may further rotate or turn those slices or segments to view in detail. In the present invention, a three-dimensional point array includes plural dots distributed in a three-dimensional space, and each dot which is called node may be to constitute a cube unit with adjacent nodes. Multiple cube units are spliced together to represent a target 3D space. Next, in the present invention, these nodes may be evenly distributed in the 3D space for subsequential operation, simulation and analysis. It is understood that the spliced cube units in the target 3D space may have identical or similar geometric shapes and dimensions. Accordingly, the 3D point array may correspond to a 3D MRI image (the target 3D space) and the 3D MRI image may be viewed to be divided into cube units of same sizes and same dimensions each of which includes nodes as its own endpoints.

The method for establishing circuit profile of electrical field simulation for temporal interference brain stimulation and the method for forming result of electrical field simulation for temporal interference brain stimulation may be stored in a computer-readable storage medium or storage device that includes computer-executable program codes and be performed by one or more processor coupled therewith. For example, an electrical apparatus, one or more integrating electrical apparatus, or one or more local electrical apparatus together with remote cloud servo or/and database execute and perform the method for establishing circuit profile of electrical field simulation for temporal interference brain stimulation and the method for forming result of electrical field simulation for temporal interference brain stimulation.

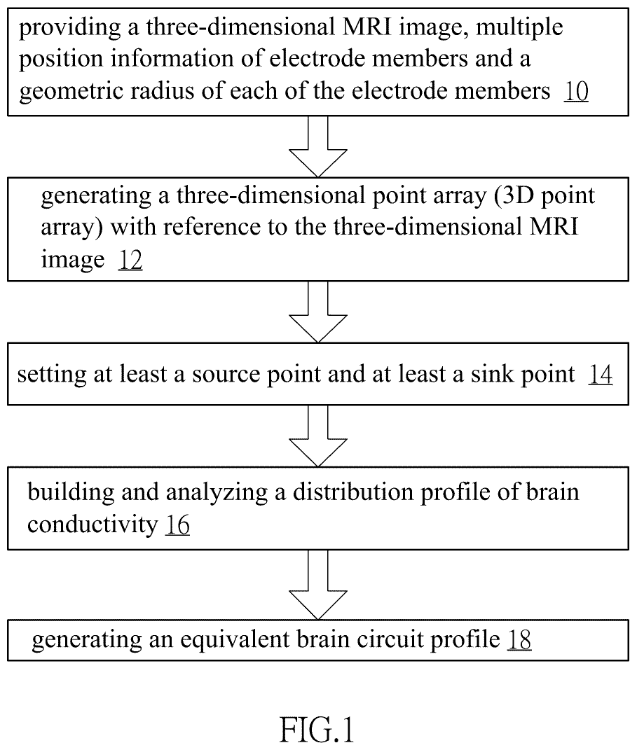

FIG. 1 is a schematic flowchart diagram illustrating an example of a method for establishing circuit profile of electrical field simulation for temporal interference brain stimulation in accordance with the present invention. Please refer to FIG. 1, the method for establishing circuit profile of electrical field simulation for temporal interference brain stimulation begins to provide a three-dimensional MRI image, multiple position information of electrode members and a geometric radius of each of the electrode members (step 10). In one embodiment, a participant's head is scanned by magnetic resonance imaging (MRI) and signals are captured to be converted to image data as a 3D MRI image of the head in the present invention. For technology of temporal interference (TI), electrode members for forming alternating current are generally mounted onto a head-wearable device and adjacent to the surface of the head. Next, position information and geometric radius of the electrode member relative to a reference point (such as a geometric center of the head-wearable device) are acquired through detecting or converting ways. It is understood that the so-called geometric radius represents a range project on the surface of head instead of restricting the physical shape or distribution of the electrode member. Furthermore, the number of electrode members is four in two sets but not limited in the present invention.

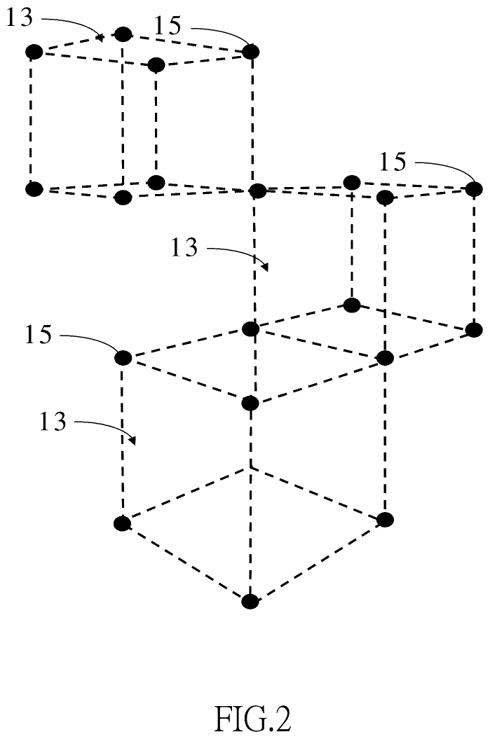

FIG. 2 is a schematic diagram illustrating a three-dimensional point array in accordance with the present invention. Please refer to FIG. 1 and FIG. 2, a three-dimensional point array (3D point array) is generated with reference to the three-dimensional MRI image (step 12). In one embodiment, the 3D MRI image of a participant may be represented as the 3D point array. The 3D point array includes plural nodes 15 on plural cube units 13, and these nodes 15 are distributed within a target space which is enclosed by the 3D MRI image. Each node 15 together with surrounding nodes 15 constitute a cube unit 13, such as a cube unit with side length 3 mm. The plural cube units 13 are spliced together to build a target space corresponding to the three-dimensional MRI image. In the present invention, the plural cube units 13 may be identical to each other to evenly distribute the nodes 15. That is, the target space corresponding to different regions of a brain is described as nodes in homogenous density. For consideration of computation and time efficiencies, the target space with evenly distributed nodes is preferred. However, the distribution density of the nodes within the target space may be modified by adjusting the sizes of the cube units or combining the cube units of different sizes for increasing or decreasing the distribution density of the nodes. Next, at least a source point and at least a sink point are set up (step 14), for example, by mapping the three-dimensional point array, the plural position information of electrode members, and the plural geometric radiuses. In one embodiment, the position information of electrode members, such as coordinates and radius, is converted by way of scale conversion to be applied onto the 3D point array. The nodes representing the electrode members can be extracted by mapping for assigning them as the source point and the sink point.

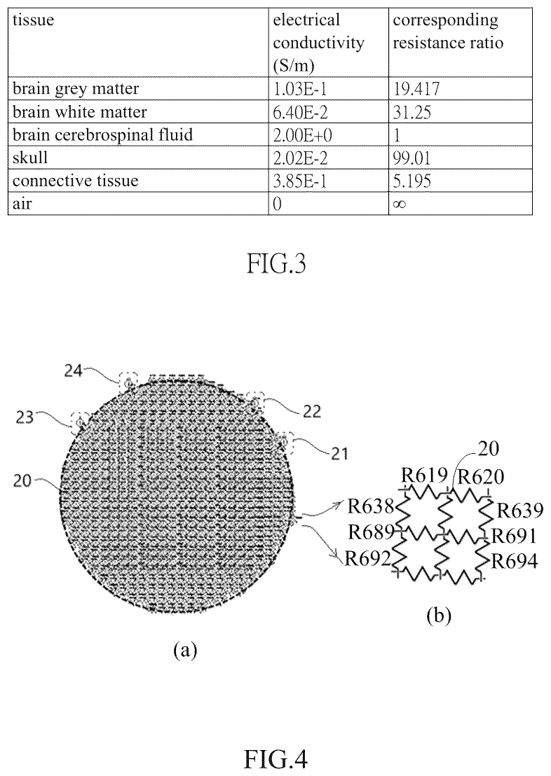

FIG. 3 is a schematic diagram illustrating some content of the database of tissue properties adopted in the present invention. FIG. 4 is a schematic diagram illustrating the distribution profile of brain conductivities in accordance with the present invention. Please refer to FIG. 1, FIG. 3 and FIG. 4, a distribution profile of brain conductivity is established and analyzed (step 16). In one embodiment, the distribution profile of brain conductivity is performed from plural resistance data corresponding to the three-dimensional MRI image, and each of the resistance data may include a resistance value between any two adjacent nodes in the three-dimensional point array. Next, in a single network of the distribution profile of brain conductivity, resistance value is calculated by way of linear combination on reference resistance data along with brain region percentages within the 3D MRI image. The reference resistance data are ones with reference to data in a database of tissue properties published by IT'IS Foundation, as shown in FIG. 3, where data is based on a frequency of 2 kHz. Please refer to FIG. 4, shown in the left picture (a), which represents the distribution profile of brain conductivity 20, and the distribution profile of brain conductivity 20 is described by a circle with reference to a sagittal plane and a diameter of 24 resistors. Shown in the right picture (b) is a zoom-out part of the left picture (b). Furthermore, a current sink 21 and a current source 22 constitute a pair of electrodes with a frequency of 2.01 kHz for example. A current sink 23 and a current source 24 constitute another pair of electrodes with a frequency of 2 kHz. Next, R619, R620, R638, R639, R689, R691, R692 and R694 respectively represent different resistors with a resistance value of 1 ohm when a brain medium is homogenous.

One of the features in the present invention is to generate the distribution profile of brain conductivity on which SPICE software may proceed an alternating current analysis to acquire a simulation result of electrical field for temporal interference brain stimulation. In the present invention, the distribution profile of brain conductivity puts weight on the calculation of voltage and current but ignores the induced electrical field caused by the changing magnetic field. Thus, the distribution profile of brain conductivity in the present invention is appropriate for the operation of the electrostatic field and semi-quasi electric field. The electrostatic field may be analyzed with direct current (the frequency is zero), and the semi-quasi electric field can be analyzed by using alternating current with frequencies of 0.001 Hz to 100 kHz. As material characteristics of different regions of brain differ, the distribution profile of brain conductivity is beneficial to match with the brain of the participant by modifying resistors of different resistances and generate a personal distribution profile of brain conductivity for any participant. A probable situation is, the brain of the participant may change with time, disease or therapy and can be captured by 3D MRI image, then the method of the present invention may adjust resistances with a circuit simulation software to correspond to the 3D MRI image and generate a present or updated distribution profile of brain conductivity for use. Furthermore, due to using cube units in the method of the present invention, an even slicing or segmentation process on the network of the brain may be achieved. A voxel of a cube unit as a basic unit is adopted in the 3D MRI image of the present invention, and then the cube units with resistors may be processed by SPICE circuit simulation software for current analysis. Thus, it is beneficial to provide a more convenient and precise method for establishing a circuit profile of electrical field simulation for temporal interference brain stimulation. Moreover, a subsequential electrical field may be outputted with the presentation of coordinate dots and acquired without an interpolation calculation, which is suitable for image input and result output of electrical field, too.

Next, an equivalent brain circuit profile is generated (step 18) with the file extension “.lis”. In one embodiment, the distribution profile of brain conductivity may be stored as an appreciate file, such as one with the file extension “.net”, to include node information and resistance information. The file with the file extension “.net” is extracted by SPICE and performed a current analysis to acquire the file with the file extension “.lis,” which includes voltage information of nodes for the subsequential analysis of the electrical field. Accordingly, the equivalent brain circuit profile of the present invention may apply to situations involving temporal interference brain stimulation to acquire the result of electrical field simulation.

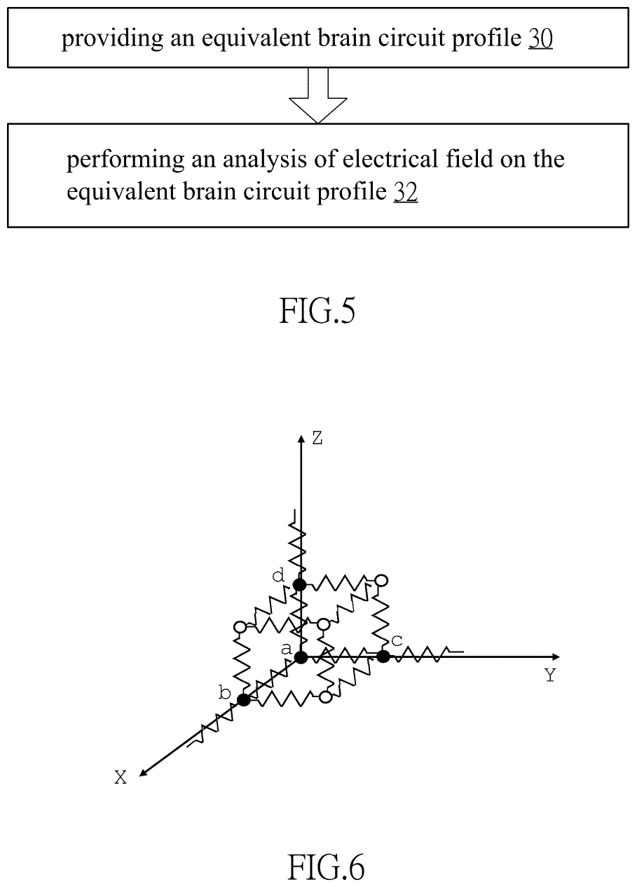

FIG. 5 is a schematic flowchart diagram illustrating a method for forming an electrical field simulation of temporal interference brain stimulation in accordance with the present invention. FIG. 6 is a schematic diagram illustrating an alternating current analysis in the method for forming an electrical field simulation of temporal interference brain stimulation in accordance with the present invention. Please refer to FIG. 2, FIG. 5 and FIG. 6, an equivalent brain circuit profile is provided (step 30), which may be acquired from the method for establishing a circuit profile of electrical field simulation for temporal interference brain stimulation. Then the equivalent brain circuit profile is performed, and an analysis of the electrical field (step 32) is performed with a program, such as Python (programming language), to acquire a result after completion of temporal interference brain stimulation or an effect evaluation. Shown in FIG. 6, within a space of orthogonal coordinates (X, Y and Z axes), eight nodes in a cube unit are respectively represented as four white dots and four black dots, and the block dots a, b, c and d respectively with four voltage Va, Vb, Vc and Vd are used for illustration of voltage. Regarding the cube unit in FIG. 6, a preset distance between any two adjacent dots is 3 mm, an electric field Ex in the direction of X axis is acquired by calculating a voltage difference between dot a and dot b(Va minus Vb) and dividing the voltage difference by 3 mm, an electric field Ey in the direction of Y axis is acquired by calculating a voltage difference between dot a and dot c(Va minus Vc) and dividing the voltage difference by 3 mm, and an electric field Ez in the direction of Z axis is acquired by calculating a voltage difference between dot a and dot d(Va minus Vd) and dividing the voltage difference by 3 mm. That is, in one embodiment, any two adjacent nodes 15 are performed with voltage calculation for acquiring the values of electrical fields in three directions (X, Y and Z axes in the orthogonal coordinates system), which may represent the interfered regions of the participant's brain as electrical field for temporal interference brain stimulation is imposed onto the participant. It is understood that those values of electrical field results may be further performed by standardization and envelope amplitude calculation to acquire the simulation results of electrical field simulation.

The above description is merely exemplary of the prefeed embodiments of the present invention and should not be construed as limiting the scope of the invention. Any modifications, equivalents, substitutions, or improvements made within the spirit and principles of the present invention should be encompassed within the scope of protection of the invention.

What is claimed is:

1. A method for establishing a circuit profile of electrical field simulation for temporal interference brain stimulation, comprising:
providing a three-dimensional MRI image, multiple position information of electrode members, and a geometric radius of each of the electrode members;
generating a three-dimensional point array with reference to the three-dimensional MRI image, wherein the three-dimensional point array comprises plural nodes on plural cube units, and the plural cube units are spliced together to correspond to the three-dimensional MRI image;
set at least a source point and at least a sink point by mapping the three-dimensional point array, the plural position information of electrode members and the plural geometric radiuses to assign some of the plural nodes as at least the source point and at least the sink point;
establishing a distribution profile of brain conductivity, wherein the distribution profile of brain conductivity comprises plural resistance data corresponding to the three-dimensional MRI image, and each of the resistance data comprises a resistance value between any two adjacent nodes in the three-dimensional point array; and
acquiring an equivalent brain circuit profile by analyzing currents for the distribution profile of brain conductivities, the source point and the sink point with a circuit simulation software.

2. The method for establishing a circuit profile of electrical field simulation for temporal interference brain stimulation according to claim 1, wherein the cube units have identical sizes.

3. The method for establishing a circuit profile of electrical field simulation for temporal interference brain stimulation according to claim 1, wherein the step of setting at least the source point and at least the sink point further comprises processing coordinate and scale conversion on the plural position information of electrode members and the plural geometric radiuses.

4. The method for establishing a circuit profile of electrical field simulation for temporal interference brain stimulation according to claim 1, wherein the circuit simulation software comprises a simulation program with integrated circuit emphasis (SPICE) software.

5. The method for establishing a circuit profile of electrical field simulation for temporal interference brain stimulation according to claim 4, wherein the simulation program with integrated circuit emphasis (SPICE) software executes current analysis with frequencies from 0 Hz to 100 kHz on the distribution profile of brain conductivities.

6. The method for establishing a circuit profile of electrical field simulation for temporal interference brain stimulation according to claim 1, wherein the cube units have different sizes.

7. A formation method for simulation results of an electrical field for temporal interference brain stimulation, comprising:
providing a three-dimensional MRI image, multiple position information of electrode members and a geometric radius of each of the electrode members;
generating a three-dimensional point array with reference to the three-dimensional MRI image, wherein the three-dimensional point array comprises plural nodes on plural cube units, and the plural cube units are spliced together to correspond to the three-dimensional MRI image;
set at least a source point and at least a sink point by mapping the three-dimensional point array, the plural position information of electrode members and the plural geometric radiuses to assign some of the plural nodes as the source point and the sink point;
establishing a distribution profile of brain conductivity, wherein the distribution profile of brain conductivity comprises plural resistance data corresponding to the three-dimensional MRI image, and each of the resistance data comprises a resistance value between any two adjacent nodes in the three-dimensional point array;
acquiring an equivalent brain circuit profile by analyzing currents for the distribution profile of brain conductivities, the source point and the sink point with a circuit simulation software; and
doing an electrical field analysis of the equivalent brain circuit profile to acquire a simulation result of the electrical field.

8. The formation method for simulation results of an electrical field for temporal interference brain stimulation according to claim 7, wherein the step of doing the electrical field analysis comprises calculating a voltage difference between any two adjacent nodes in any one of the cube units and dividing the voltage difference by the distance of the two adjacent nodes to acquire an electrical field value.

9. The formation method for simulation results of an electrical field for temporal interference brain stimulation according to claim 7, wherein the electrode members comprise two sets of electrode pairs.

10. The formation method for simulation results of an electrical field for temporal interference brain stimulation according to claim 7, wherein the cube units have identical sizes.

11. The formation method for simulation results of an electrical field for temporal interference brain stimulation according to claim 7, wherein the step of setting at least the source point and at least the sink point further comprises processing coordinate and scale conversion on the plural position information of electrode members and the plural geometric radiuses.

12. The formation method for simulation results of an electrical field for temporal interference brain stimulation according to claim 7, wherein the circuit simulation software comprises a simulation program with integrated circuit emphasis (SPICE) software.

13. The formation method for simulation results of an electrical field for temporal interference brain stimulation according to claim 12, the simulation program with integrated circuit emphasis (SPICE) software executes current analysis with frequencies from 0 Hz to 100 kHz on the distribution profile of brain conductivities.

14. The formation method for simulation results of an electrical field for temporal interference brain stimulation according to claim 7, wherein the cube units have different sizes.

15. A computer-readable storage medium containing instructions executable by a computer for causing the computer to perform operations comprising:
providing a three-dimensional MRI image, multiple position information of electrode members and a geometric radius of each of the electrode members;
generating a three-dimensional point array with reference to the three-dimensional MRI image, wherein the three-dimensional point array comprises plural nodes on plural cube units, and the plural cube units are spliced together to correspond to the three-dimensional MRI image;
set at least a source point and at least a sink point by mapping the three-dimensional point array, the plural position information of electrode members and the plural geometric radiuses to assign some of the plural nodes as the source point and the sink point;
establishing a distribution profile of brain conductivity, wherein the distribution profile of brain conductivity comprises plural resistance data corresponding to the three-dimensional MRI image, and each of the resistance data comprises a resistance value between any two adjacent nodes in the three-dimensional point array; and
acquiring an equivalent brain circuit profile by analyzing currents for the distribution profile of brain conductivities, the source point and the sink point with a circuit simulation software.

16. The computer-readable storage medium according to claim 15, further comprising doing an electrical field analysis of the equivalent brain circuit profile to acquire simulation results of electrical field.

17. The computer-readable storage medium according to claim 15, wherein the cube units have identical sizes.

18. The computer-readable storage medium according to claim 15, wherein the step of setting at least a source point and at least a sink point further comprises processing coordinate and scale conversion on the plural position information of electrode members and the plural geometric radiuses.

19. The computer-readable storage medium according to claim 15, wherein the circuit simulation software comprises a simulation program with integrated circuit emphasis (SPICE) software that executes current analysis with frequencies from 0 Hz to 100 kHz on the distribution profile of brain conductivities.

20. The computer-readable storage medium according to claim 15, wherein the cube units have different sizes.
